(19) United States
(12) Patent Application Publication
(10) Pub. No.: US 2025/0344763 A1
(43) Pub. Date: Nov. 13, 2025

(54) Other Than Cannabinoids, Flavonoids, and Terpenes

(71) Applicant: Raymond Louis Larson, Aliso Viejo, CA (US)

(72) Inventor: Raymond Louis Larson, Aliso Viejo, CA (US)

(21) Appl. No.: 16/873,820

(22) Filed: Jul. 17, 2020

Related U.S. Application Data
(60) Provisional application No. 62/921,899, filed on Jul. 15, 2019.
(60) Provisional application No. 63/100,380, filed on Mar. 11, 2020.
(60) Provisional application No. 63/101,278, filed on Apr. 24, 2020.

(51) Int. Cl. A24F 40/42; A24F 40/10; A61K 31/05; A61K 31/352

(52) U.S. Cl. CPC A24F 40/42; A24F 40/10; A61K 31/05; A61K 31/352

(57) ABSTRACT

A pharmaceutical composition and administration apparatus includes a portable powered vaporizer with a mouthpiece, smokeless vaporizing element, and a removable chamber. The chamber contains a composition comprising tetrahydrocannabinol (THC), cannabidiol (CBD), FCC grade ethanol, flavoring, pharmaceutical grade nicotine, USP grade aqueous glycerine, USP/EP grade propylene glycol, and USP grade vegetable glycerine. In various formulas the THC may be at concentrations of 1-30%, 30-60% and 60-99%, while CBD concentrations may be 0-15% including other cannabinoids derived from extraction.

This application benefits from U.S. provisional patent applications: 62/921,899, 63/100,380, 63/101,278. Priority is claimed to all of said applications, each of which is incorporated by reference herein in their entirety.

### BACKGROUND

Marijuana plant varieties xCannabis setiva, Cannabis indica, Cannabis rederalis, etc.) contain a variety of compounds, the major psychoactive compound being tetrahydrocannabinol (THC). Other compounds include a variety of additional cannabinoids, including cannabidiol (CBD), which is less psychoactive than THC and believed to have a wider scope of medical applications. Numerous publications report analgesic, antiemetic, and antiglaucomal effects, among others as resulting from CBD. While typically used recreationally, THC has also been shown to be an effective analgesic, antiemetic, and useful in treating nausea and attendant effects of cancer chemotherapy.

Administration of cannabinoids has evolved over time from the inhalation of marijuana combustion byproducts (i.e., smoking) to oral consumption of marijuana when combined with foods, or the oral consumption of cannabinoid compounds (such as THC and other cannabinoids) in extracted pill form. Additionally, transdermal patches, as discussed in U.S. Pat. No. 6,503,532 have been developed and used in the art. These methods of administration are disfavored however.

When inhaling combustion byproducts, users cannot regulate the percentage of individual cannabinoids entering the lungs. Additionally, undesirable irritating, and potentially toxic or carcinogenic are produced and inhaled as well. The use of orally consumed products such as pills allows users to adjust the quantity of a given cannabinoid ingested, but is disfavored due to the delay of cannabinoid effects caused by the digestive system. Additionally, many marijuana users prefer the mimicry of smoking conferred by inhalation for personal and social reasons. While a transdermal patch may be an effective way of time-releasing a known quantity of particular cannabinoids into the blood stream, this method suffers from drawbacks similar to orally ingested pills, including a lack of social interaction.

For these reasons, there is a need for a cannabinoid composition possessing a predetermined quantity of THC and CBD (in addition to other cannabinoids) for users to obtain a desired recreational, medical or combined effect. There is also a need for an administration technique that allows users to control the quantity of cannabinoids administered through an inhalation apparatus that avoids simple combustion.

### SUMMARY

Presented is a formulation of cannabinoid and a smokeless administration of use through vaporization in a portable and rechargeable delivery system. The cannabinoids are mixed with several vegetable glycerols including propylene glycol, among other components. Several cannabinoid formulations with varying levels of THC and CBD are contemplated, and preferably will be contained in a cartridge. In this manner, cartridges can be interchangeable with other cartridges depending on a desired effect.

Two embodiments of cartridges are contemplated, a disposable cartridge and a refillable cartridge. In the instance of a refillable cartridge, a syringe may accompany the delivery system for refilling the cartridge. This is due to the high viscosity of high THC/CBD percentage formulations and the attendant difficulty of transferring them from a source vessel into a refillable cartridge.

### BRIEF DESCRIPTION OF THE FIGURES

FIG. 1 shows a disposable smokeless “e-cigarette” type vaporizing apparatus.

FIG. 2 shows a smokeless “e-cigarette” type vaporizing apparatus having a disposable cartridge.

FIG. 3 shows a disassembled smokeless “e-cigarette” type vaporizing apparatus having a refillable cartridge.

FIG. 4 shows a syringe used to refill the cartridge of a refillable “e-cigarette” type vaporizing apparatus.

FIG. 5 shows chemical formula for Tetrahydrocannabinol.

FIG. 6 shows the chemical formula for Cannabidiol.

### DESCRIPTION

An improved pharmaceutical composition and administration apparatus is disclosed. The administration apparatus comprising a portable powered vaporizing apparatus having a mouthpiece, a smokeless vaporizing element, and a removable chamber. Portable vaporizers of this variety are typically known as e-cigarettes, which have a heating element for vaporizing nicotine and nicotine-containing oils. In the embodiments disclosed herein, a heating element calibrated for the use cannabinoids is contemplated.

When prepared for use, the chamber contains a cannabinoid composition comprising tetrahydrocannabinol (THC), cannabidiol (CBD), FCC grade ethanol, flavoring, pharmaceutical grade nicotine, USP grade aqueous glycerine, USP/EP grade propylene glycol, and USP grade vegetable glycerine.

Preferably a food grade ethanol and flavoring is used in the composition. Additionally, in one preferred embodiment, the nicotine may include USP grade nicotine extract, and may be extracted from organic sources. Prior to manufacturing

In one preferred embodiment the food grade glycerin will comprise a 99.7% USP Grade Glycerine having 99.70% Glycerol content, 0.3% maximum moisture content, 10 ppm max chlorides, 10 max (APHA) coloring, a minimum specific gravity of 1.2612, 20 ppm max, 5 ppm max heavy metals, 30 ppm max chlorinated compounds, I 00 ppm max residue on ignition, 1.000% max fatty acid and esters, 0.5% water, and assayed at between 99.0 to 101.0% (on a dry basis). Additionally the food grade glycerine passes tests for DEG and related compounds and organic volatile impurities. In another embodiment pure vegetable glycerin may be used.

Preferably the propylene glycol selected for usage will be pharmaceutical grade propylene glycol with a specified purity greater than 99.8%, and in compliance with United States Pharmacopeia (USP), European Pharmacopeia (EP), Japanese Pharmacopeia (JP) and Food Chemical Codex (FCC) standards.

Several formulas are contemplated when formulating the pharmaceutical composition. In all cases, where 0% is listed, it is understood that any amount under the maximum percentage down to greater than 0% is intended, including trace amounts. A first formula includes a 30% THC:

| | |
|---|---|
| 0% to 30% | Tetrahydrocannabinol (THC) |
| 0% to 15% | Remaining cannabinoids including cannabidiol (CBD) |
| 0% to 20% | FCC grade ethanol |
| 0% to 04% | Food grade flavoring |
| 0% to 03% | Pharmaceutical grade nicotine |
| 0% to 20% | USP grade acqueous glycerine |
| 0% to 70% | USP/EP grade propylene glycol |
| 0% to 25% | USP grade vegetable glycerine |

A second formula combines known cannabinoids THC and CBD at specific concentrations:

| | |
|---|---|
| 0% to 30% | Tetrahydrocannabinol (THC) |
| 0% to 15% | Cannabidiol (CBD) |
| 0% to 15% | Remaining cannabinoids excluding cannabidiol (CBD) |
| 0% to 20% | FCC grade ethanol |
| 0% to 04% | Food grade flavoring |
| 0% to 03% | Pharmaceutical grade nicotine |
| 0% to 20% | USP grade acqueous glycerine |
| 0% to 70% | USP/EP grade propylene glycol |
| 0% to 25% | USP grade vegetable glycerine |

A third formula combines employs a greater percentage of THC along with other

known cannabinoids including cannabidiol at a lower percentage, and reduced percentages of acqueous glycerine and propylene glycol:

| | |
|---|---|
| 30% to 60% | Tetrahydrocannabinol (THC) |
| 0% to 15% | Remaining cannabinoids including cannabidiol (CBD) |
| 0% to 20% | FCC grade ethanol |
| 0% to 04% | Food grade flavoring |
| 0% to 03% | Pharmaceutical grade nicotine |
| 0% to 10% | USP grade acqueous glycerine |
| 0% to 60% | USP/EP grade propylene glycol |
| 0% to 25% | USP grade vegetable glycerine |

A fourth formula comprises the higher percentage of THC but with a known

quantity of CBD in addition to remaining cannabinoids. As with the previous formula, the acqueous glycerine and propylene glycol are reduced, but in this case including the USP vegetable grade glycerine as well.

| | |
|---|---|
| 30% to 60% | Tetrahydrocannabinol (THC) |
| 15% to 30% | Cannabidiol (CBD) |
| 0% to 15% | Remaining cannabinoids excluding cannabidiol (CBD) |
| 0% to 20% | FCC grade ethanol |
| 0% to 04% | Food grade flavoring |
| 0% to 03% | Pharmaceutical grade nicotine |
| 0% to 10% | USP grade acqueous glycerine |
| 0% to 60% | USP/EP grade propylene glycol |
| 0% to 15% | USP grade vegetable glycerine |

A fifth formula comprises the maximum percentage of THC. With this formula, it is intended that either replaceable cartridges or a syringe will be used due to the higher viscosity of the pharmaceutical composition. Additionally, in this formulation, a much reduced quantity of acqueous glycerine, propylene glycol and vegetable glycerine is used:

| | |
|---|---|
| 60% to 99% | Tetrahydrocannabinol (THC) |
| 0% to 10% | Remaining cannabinoids including cannabidiol (CBD) |
| 0% to 20% | FCC grade ethanol |
| 0% to 04% | Food grade flavoring |
| 0% to 03% | Pharmaceutical grade nicotine |
| 0% to 10% | USP grade acqueous glycerine |
| 0% to 40% | USP/EP grade propylene glycol |
| 0% to 10% | USP grade vegetable glycerine |

The sixth formula adds a known quantity of CBD to the high concentration of THC in the formula. Due to the high percentage of THC and CBD, the remaining cannabinoids are reduced as well as the acqueous glycerine, propylene glycol and vegetable glycerine:

| | |
|---|---|
| 60% to 99% | Tetrahydrocannabinol (THC) |
| 0% to 15% | Cannabidiol (CBD) |
| 0% to 10% | Remaining cannabinoids including cannabidiol (CBD) |
| 0% to 20% | FCC grade ethanol |
| 0% to 04% | Food grade flavoring |
| 0% to 03% | Pharmaceutical grade nicotine |
| 0% to 10% | USP grade acqueous glycerine |
| 0% to 40% | USP/EP grade propylene glycol |
| 0% to 10% | USP grade vegetable glycerine |

In all embodiments it is preferable to have an administration apparatus that is a vaporizer having a mouth piece, a chamber for holding a quantity of the pharmaceutical composition, and a battery powered heating element. A preferable administration apparatus may include an “e-cigarette” type apparatus in which a battery powered heating element is combined with a chamber for holding a preferred cannabinoid formulation. The cannabinoid formulation is drawn from the chamber and heated by the heating element to a vaporization temperature at which point the vapors are inhaled in by a user.

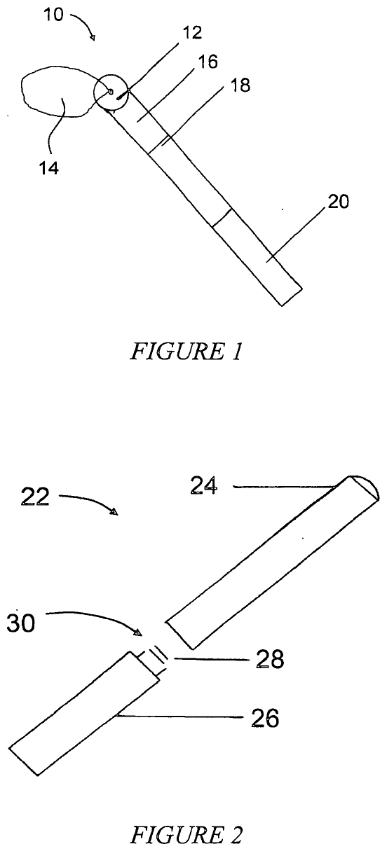

Referring to FIG. 1, one common type of preferred and inexpensive administration apparatus is a onetime use only disposable e-cigarette vaporizer 10. The disposable e-cigarette vaporizer 1 0 includes a mouthpiece 12 for extracting vaporized cannabinoids 14, the mouthpiece 12 is in fluid communication with an atomizer 16 which combines vaporized cannabinoids 14 with air drawn through the disposable e-cigarette vaporizer 10. The atomizer 16 is powered by a battery 18 adapted to power the atomizer 16 sufficiently to vaporize a quantity of cannabinoids housed in a chamber 20 in fluid communication with the atomizer 16. In this embodiment, once the chamber 20 is empty or the battery 18 dies, the entire apparatus is discarded.

Referring to FIG. 2, a second type of preferred administration apparatus includes a replaceable cartridge vaporizer 22. The replaceable cartridge vaporizer 22 includes a battery 24. A chamber 26 and atomizer 28 are connectable to the battery 24 using a threaded connection 30. In this manner, the chamber 26 may be replaceable once a quantity of cannabinoid formula housed in the chamber 26 is exhausted. This apparatus may be preferable since the battery 24 is capable of vaporizing a quantity of cannabinoid formula in multiple chambers.

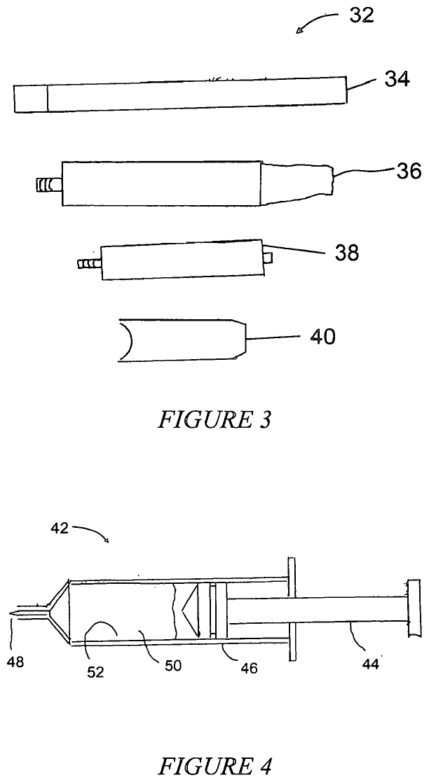

Referring to FIG. 3, a third type of preferred administration apparatus is a commonly used e-cigarette vaporizer 32. In this embodiment the components of the e-cigarette vaporizer 32 may be disassembled into a battery 34, either a disposable cartridge 36 or refillable cartridge 38, and a mouthpiece 40. The battery 34 is preferably combined with an atomizer (not shown). Also, in instances where a disposable cartridge 36 is used, the disposable cartridge 36 may have a mouthpiece incorporated therein.

Referring to FIG. 4, in certain instances, where a refillable cartridge 36 is used, the cannabinoid formulation may be to viscous for easy decanting into a refillable cartridge 36. In such an instance, a syringe 42 may be employed. It is anticipated that a typical syringe having a plunger 44, barrel 46 and needle 48 will be used, although the needle may be adapted and sized for an efficient union with the refillable cartridge 36 (FIG. 3). While the syringe 42 may be used to take up a cannabinoid mixture 50 into the barrel interior 52, it is also anticipated that syringes may be produced and purchased pre-loaded with a cannabinoid mixture 50, ready for introduction into an empty cartridge 36. In this manner cartridges may be easily and effectively refilled with a desired cannabinoid formulation.

When properly administered, the pharmaceutical composition of the present invention may be useful for both recreational and medical purposes. Medical uses may include, but are not limited to, treating pain, nausea, loss of appetite (particularly nausea associated with chemotherapy in cancer treatments), glaucoma, arthritis, dementia, multiple sclerosis, and for deterring weight loss.

While the apparatus and associated formulas have been described in detail with reference to specific embodiments thereof, it will be apparent to one skilled in the art that various changes and modifications can be made therein without departing from the spirit and scope thereof. Thus, it is intended that the present description cover the modifications and variations of the invention provided they come within the scope of the appended claims and their equivalents.

In this invention it is imperative regarding the avoidance and/or exclusion of both: THC and CBD. However this invention as a formulary includes:
ALL other cannabinoids, except and specifically CBD & THC as the 2 excluded cannabinoids. Currently, well over a hundred noted cannabinoids have been discovered in Cannabis/Hemp.

This invention is relative to ALL infused products currently known and also those not yet innovated that can be for all living bodies: humans, pets, mammals, etc. for consumption. Some delivery examples of this provisional formulary given yet not limited to: edibles, skin care, essential oil mixtures, sublingual & spray forms, pills, bath and body lotions, shampoo, topically, IV, inhalation, nasal delivery, suppository, beverages (Coffee/tea/water/beer/alcohol, etc.) whether in low or high viscous forms. The estimated sufficient rage I feel would be accommodating is greater than zero (0) % and up to 20% by weight.

Some examples, perhaps, would be:
1) Greater than zero (0) % by wet. to 20% wt. cannabinoids excluding CBD.
2) Greater than (0) % by wt. to 20% by wt. other than THC and CBD in a cannabinoid formulary.
3) ALL cannabinoids except for THC and CBD in an essential oil formulary.

Basically, any and all other cannabinoid formulas/inventions found to contain THC and or CBD however with this provisional filing/invention, THC and/or CBD are specifically excluded.

Terpene formulas and flavonoid formulas will be excluding CBD and/or THC. Terpenes, Flavonoids, and Cannabinoids in all formulations from all 3 lists: A, B, and C can be one or more ingredients taken from any of the mentioned or added lists. Formulas maybe excluding one or more of: A, B, C to any one formula such as Myrcene & CBD and/or THC. Formulas maybe also be excluding Flavonoids and/or CBD, THC in % of Terpenes from greater than zero (0) to 30%. Formulas may have synthetic or any other plant Terpene or Flavonoid included.

This invention is relative to ALL infused products currently known and also those not yet innovated that can be for all living bodies, humans, pets, mammals, etc. for consumption. Some delivery examples of this provisional formulary given yet not limited to edibles, skin care, essential oil mixtures, sublingual & spray forms, pills bath and body lotions, shampoo, topically, IV inhalation, nasal delivery, suppository, beverages (coffee/tea/water/beer/alcohol, etc.) whether in low or high viscous forms. The estimated sufficient range I feel would be accommodating is greater than zero (0) % and up to 20% by weight.

### Example of Formulary

0%-30% wt THC and 30%-60% wt THC or 60%-99% wt THC
0%-15% wt CBD
One or more of remaining cannabinoids greater than 0% wt to 20% wt chosen from the C list, excluding cannabidiol (CBD) and/or THC
Greater than 0% wt to 15% wt of the C list and a terpene from list A other
than CBD and/or THC and myrcene
0% wt-20% FCC ethanol or alcohol
0% wt-5% wt Food Grade Flavoring
0% wt-5% wt Pharmaceutical grade nicotine
0% wt-25% USP grade aqueous glycerin
0% wt-40% wt USP/EP grade glycol
0% wt-60% wt USP grade vegetable glycerin
0% wt-70% wt coconut oil
0% wt-70% wt essential oils
0% wt-15% wt terpenes
0% wt-15% wt flavonoids
15% wt-30% wt CBD
30% wt-60% wt CBD
1% wt-30% wt selected item(s) on list(s)
1% wt-30% wt selected terpene(s)/flavonoids

This invention deals with compositions using NO THC (Tetrahydrocannabinol). There is only one symbol, as shown in FIG. 5, that exits in the chemistry world. You don't have to be a person skilled in the art to see the difference. A person skilled in the art can see by using NO CBD compound symbol, as shown in FIG. 6, in any part of the formula.
Sample 1: A liquid composition: 1.5% wt-15% wt Cannabinoids other than TIIC, CBD, and Trace-3% Nicotine.
Exhibit-B shows THC and CBD separated. We show some of the other Cannabinoids together a person skilled in the art can see the different things the other Cannabinoids do on their own. This is not a 103 issue.
Exhibit-B1 shows only THC and CBD.
Exhibit B-2 shows NO THC and/or CBD.
Sample 2: A liquid composition 1.1% wt.-15% wt. Cannabinoids OTHER THAN, THC and/or CBD
Sample 3: A liquid composition trace −15% Cannabinoids other than THC and/or CBD

This invention presents several formulations with most of them using OTHER THAN THC and CBD. Within the formula(s), the inventor has added or subtracted percentages of cannabis related compounds. Example: CBN % wt. 0% wt-50% wt. This is an example of one of the cannabinoids of which will be used. Besides cannabinoids, Terpenes, Flavonoids, and other plant based matter including also synthetics.

Formulas will include: Water, Carbonated Water, Water Soluble matter, Hops, Ethanol, Virtually All Beverages, Nicotine infused pills/cough drops, Beer, Wine, and any new or newly discovered infusible ideas.

This invention uses ALL 3 of the compounds: Cannabinoids, Terpenes, Flavonoids other than THC and CBD for ALL 3 categories. Oral and topical will be also used. Also used will be virtually all consumables like, Beverages. Also, skin care products The use of pills and other delivery methods used in this invention. There are over 100 cannabinoids discovered thus far, however there are many undiscovered drugs that are still undiscovered thus, will be formulated. The amounts of OTHER THAN THC and CBD are very low in Cannabis (Marijuana and Hemp). I am targeting to increase or decrease the amount of each compound including Flavonoids and Terpenes. With the high cost of these rare (OTHER THAN THC/CBD) compounds in each plant like CBN for instance today is $25,000 per Kilo VS steel at $6.00 per Kilo. Some formulas may EXCLUDE one or more of ALL cannabinoids, Flavonoids, and Terpenes.

Below is a list of possible ingredients that can be included in the present invention:
1) 0% wt.-95% wt. Water Soluble
2) 0% wt.-35% wt. Hops
3) 0% wt.-95% wt. Carbonated Water
4) 0% wt.-98% wt. Water
5) 0% wt.-35% wt. Malts
6) 0% wt.-20% wt. Electrolytes
7) 0% wt.-10% wt. Caffeine
8) 0% wt.-20% wt. Melatonin
9) 0% wt.-30% wt. THC
10) 30% wt.-60% wt. THC
11) 60% wt.-99% wt. THC
12) 0% wt.-30% wt. CBD
13) 30% wt.-60′% wt. CBD
14) 60% wt.-99% wt. CBD
15) 0% wt.-20% wt. Remaining Cannabinoids Excluding CBD
16) 0% wt.-30% wt. FCC grade ethanol
17) 0% wt.-4% wt. Food grade flavoring
18) 0% wt.-5% wt Nicotine
19) 0% wt.-80% wt. USP aqueous glycerin
20) 0% wt-80% wt. USP/EP grade propylene glycol
21) 0% wt.-80% wt. USP grade vegetable glycerin
22) 0% wt.-30% wt. Cannabinoids and/or Flavonoids and/or Terpenes Other than any individual compositions.

1-174 (canceled)

175. A composition of cannabinvids, terpenes, flavonoids comprising:
Greater than 0% wt.-50% wt. CBN Other Than CBD and a group consisting of cannabinoids two or more Cannabinoids with Tetrahydro Can-type (THC), with two or more Cannabinodiol CBND} with one or more Cannabitriol-type (CBT), two or more Cannabigerol-type (CBG) with two or more Cannabichromene-type (CBC), with two or more Cannabiabinol-type (CBN), with two or more Cannabinoids; total of combined cannabinoids selected from the group consisting of: CBN-C_{z}, CBD-C₄, CBCV-C₃, CBND-C₅, CBND-C₃, CBN-C₁, CBN-C₅, CBD-C₁, CBDV-C₃, CBCC₅, CBDA-C₅, CBDM-C₅, CBO-C₅, CBDM-C₅, CBNM-C₅, CBT-C₅, (E)-CBG-C₅, (E)-CBGM-C₅A, (Z)-CBG₅A, (E)-CBGV-C₃, (E)-CBGA-C₅A, (E)-CBGAM-C₅A, (E)-CBGB-C3A, Delta-9-THC-C₅, Delta-9-THC-C₄, Delta-9-THC-CI, Delta-9-THC-C3, Delta-9-THCA-C₅A, Delta-9-THCA-C₅B, OTHC, CBG, CBN,CBC, CBN-C4,CBN-C2,CBN-C1,CBNA-C5A, (E)CBGA-C5A,(E)-CBG-C5,CBC-C5,CBCA-C5A and CBCVA-C3A;
greater than 0% wt.-30% wt. and a group consisting of; two or more terpenes/two or more Flavonoids, one or more Flavanones, one or more Flavonas, one or more Flavouols,one or more Isoflavonoids from the group consisting of fisetin, chrysin, baicalein, naringenin, epicatechin, pelargonidin, Hesperetin, Naringenin, malvidin, sabinene hydrate, (−)-isospulegol, genistein, isoborneol, nerol, r-(+)-pulegone, geranyl acetate, fenchol, cedrol, myrcene, limonene, linalool, a pinene fenchol, Glycitoin, Genistein fencliul, borneol, terpineol, menthol, Quercetin, Galilate, Catechins and 3 Carene,
wherein the composition is a beverage, and
wherein the cannabinoids are carbonated water soluble.

176. The composition according to claim 175,
wherein the composition comprises two or more terpenes/two or more flavonoids and one or more Flavones selected from the group consisting of fisetin, chrysin, baicalein, naringeni, epicatechin, pelargonidin, malvidin, inyrcene, limonene, a pine, nerol, cedrol, frenchone, menthol, terpingol borneol, Chrysin, Baicaicin, Apigenin
and glycitein, of more than 1%. wL-30% wt. and
0% wt.-30% wt. of alcohol
0% wt.-35 % wt. Hops

177. composition according to claim 175 further comprising:
at least 2 or more terpenes/at least 2 or more flavonoids and one or more Flavanones from the group consisting of: sabinene hydrate, (−)-isopulegol, isoburn cedol, nerol, r-(+)-pulegone, geranyl acetate, Naringenin, Hesperidin, myrcene limonene, linalool, frenchone, menthol, borneol, a pinen, chrysin, nuringenin fenchol, and cedrol from more than 0% wt.-30% wt.;
0% by wt. cannabinoids; and
0% wt.-30% wt. of alcohol
0% wt.-35 % wt. of hops.

178. The composition according to claim 175 further comprising: wherein
At least two terpenes/at least two Flavonoids and one or more Flavonols and one or more Flavones from the group consisting of: sabinene hydrate, isoborneol, nerol, r-(+)-pulegone, geranyl acetate, Quercetin, Galangin, Fisetin, Myricetin, Kaempterol, Catechins, Chrysin and cedrol from more than 1% wt.-30 % wt.
0% wt.-cannabinoids,
0% wt.-30% wt. alcohol,
0% wt.-35 % wt. Hops.

179. The composition according to claim 175 further comprising:
At least two terpenes/at least two flavonoids the group consisting of: sabinene hydrate, (−)-isopulegol, isoborneol, nerol, r-(+)-pulegone, geranyl acetate, fenchol cedrol, linalool from less than 1% wt. 30% wt.

180. composition according to claim 175,
wherein a combination of cannabinoids are
wherein the composition comprises two or more terpenes/Flavonoids from the group consisting of: sabinene hydrate, (−)-isopulegol, isoborneol, nerol, r-(+)-pulegone, geranyl acetate, fenchol and limonene, linalool and nerol are less than 1% wt.-30 % wt.

181. composition according to claim 175 further compromising wherein a combination of the cannabinoids is other than cannabigerol-type(CBG) and the Remaining cannabinoids greater than 0%-30% wt.

182. composition according to claim 175.
wherein a combination of cannabinoids are greater than 0% wt.-30% wt., and
wherein the composition comprises two or more terpenes/two or more flavonoids and one or more Flavonols from the group consisting of: sabinene hydrate, (−)-isopulegol, Quercetin, isoborneol, nerol, r-(+)-pulegone, geranyl acetate, fenchol, cedrol, myrcene, menthol, borneol, limonene, linalool, a pinen, fisetin, chrysin, baicalein, naringenin, epicatechin pelargonidin, malvidin, and glycitein of greater than 0% wt.-30% wt. and 0% wt.-35 % wt. Hops
0% wt.-30% wt. alcohol.

183. composition according to claim 175, wherein the composition comprises selected from the group consisting of: and wherein a combination of cannabinoids are other than THC and the remaining Tetrahydro Can-type cannabinoids greater than 0%-30% wt., and
wherein the composition comprises two or more terpenes/two or more Flavonoids and one or more Flavonols and one or more Flavonols selected from the group consisting of: sabinene hydrate, (−)-isospulegol, isoborneol, nerol,r-(+)-pulegone, geranyl acetate, myrcene, terpineol, fenchol, quercetin, galiate, myricetin and cedrol, fisetin, chrysin, baicalein, naringenin, pelargonidin, malvidin, and glycitein, of at least greater than 0% wt-30% wt. 0% wt. of THC
0% WT. cannabinoids,
0% wt.-30% wt. alcohol
0% wt.-35 % wt. hops

184. A composition of cannabinoids, flavonoids and terpenes comprising:
Greater than 0% wt.-50 % wt. of CBG cannabinoids and The Group consisting of Cannabinol-type(CBN) of two or more and-type, (CBND) with one or more cannabinoid, The Group Cannabigerol type (CBG) with two or more cannabinoids,The Group Cannabichromene-type(CBC) with two or more cannabinolds andThe Group Cannabitriol-type(CBT) with one or more cannabinoids and Tetrahydro Can-Type (THC) with two or more cannabinolds; selected from the group
wherein the cannabinoids are selected from the group consisting of: CBN-C₂, CBD-C₄, CBCV-C₃, CBND-C₅, CBND-C₃, CBN-C₁, CBN-C₅, CBD-C₁, CBDVC₃, CBCC₃, CDDA-C₅, CBDM-C₅, CBO-C₅, CBDM-C₅, CDNM-C₅, CBT-C₅, (E)-CBG-C₅, (E)CBGM-C₅A, (Z)-CBG₅A. (E)-CBGV-C₃, (E)-CBGA-C₅A, (E)-CDGAM-C₅A, (E)-CBGVA-C₃A, Delta-9-THC-C₅, Delta-9-THC-C₄, Delta-9-THC-C1, Delta-9-THC-C3, Delta-9-THCA-C₅A, Delta-9-THCA-C₅B, CBDVA, CBGA,,OTHC, CBG, CBN, CBC,CBN-C4,CBN-C3,CBN-C2,CBN-C1,CBCV-C3,CBCA-C5A
greater than 0% wt.-30% wt.consisting of two or more terpenes/two or more flavonoids, one or more Flavonols,one or more Flavones, selected from a group consisting of: fenchol, (−)-isopulegol, isoborneol, nerol, r-(+)-pulegone, geranyl acetate, myrcene, limonene, linalool, pinene, cedrol, Quercetin, Gaiangin, Myricetin,
at galangin, fisetin, chrysin, baicalein, naringenin, epicatechin, gallate, pelargonidin, malvidin, and glycitein.
Wherein the compositions may be synthetic compounds.

185. The composition according to claim 184,
wherein the composition comprises a combination of cannabinoids is other than cannabinol-type(CBN) all remaining cannabinoids
wherein the composition is greater than 0% wL.-30%.

186. composition according to claim 184
wherein the composition comprises two or more terpens/two or more Flavonoids selected from the group consisting of galangin, fisetin, chrysin, haicalein,, naringenin,
epicatechin, gallate, pelargonidin, malvidlin, and glyoitoin, wherein the two or more flavonoids/two or more terpenes are great than 0% wt.-30% wt.

187. composition according to claim 184, further comprising: selected from the group consisting of: wherein a combination of cannabinoid is greater than 0% wt.-30% wt., and Wherein the composition is an edible with no synthetic compound.

188. The composition according to claim 184 further from the group consisting of: wherein a combination of cannabinoids other than(CBC) type all remaining cannabinoids is greater than 0% wt. 30% wt. Wherein the composition is an edible.
wherein the composition comprises Wherein the compounds are greater than 0% wt-30% wt. the composition is pet edible.

189. composition of cannabinoids, flavonoids and terpenes comprising:
Greater than 0% wt.-50 % wt. CBG cannabinoids and consisting to include CBGtwo or more cannabinoids of Cannabinol-type(CBN) with two or more cannabinoids and or Cannabigerol type (CBG) with two or more cannabinoids and or Cannabichromene-type(CBC) with two or more cannabinoide and or Cannabinol-type(CBT) with one or more cannabinoide Cannabinol-type(CBND) with one or more cannabinoids; selected from a group consisting of: CBN-C₂, CBD-C₄CBD. CBCV-C₃. CBND-C₅, CBND C₃, CBN-C₁, CBN-C₅, CBD-C₁, CBD, CRDV-C₃, CBCC₅, CBDA-C₅, CBDM-C₅, CBO-C₅, CBDM-C₅, CBNMC₅, CBT-C₅, (E)-CBG-C₅, (F CBGM-C₅A, (Z)-CBG₅A, (E)-CBGV-C₃, (E)-CBGA-C₅A, (F)-CBGAM-C₅A, (E)-CBGB-C3A, Delta-9-TIICC₅, THC, Delta-9-THC-C₄, Delta-9-THC-C1. Delta-9-THC-C3, Delta-9-THCA-C₅A,OTHC, CBG, CBN, CBC, and Delta-9-THCA-C₅B and two or more terpenes/two or more flavonoids, one or more Flavonols, one or more Flavonols
selected from a groups consisting of: sabinene hydrate, (−)-isopulegol fenchol, isoborneol, nerol, r-(+)-pulegone, geranyl acetate, myrcene, limonene, linalool, a pinene, frenchone, cedrol, Myricetin, Quercetin, galangin, fisetin, chrysin, baicalein, naringenin, glycitein, galangin, gallate, epicatechin, pelargonidin, and malvidin. Wherein a total to include with Terpenes/Flavonoids greater than 0% wt.-30% wt.
Wherein the composition may be a synthetic compound.

190. The composition according to claim 189, further comprising: wherein the cannabinoids from claim 189 other than cannabitriol type(CDC) wherein the composition comprises at two or more terpenes/two or more flavonoids selected from the group consisting of: sabinene hydrate, (−)-isopulegol, galangin, chrysin, isoborneol, myreene, limonene, naringenin, nerol, r-(+)-pulegone, geranyl acetate.. fisetin, chrysin, myrcene, nerol, menthol, cedrol, wherein the terpenes/flavonoids are greater than 0% wt.-30% wt., and
wherein the composition is an edible for pets.

191. the composition according to claim 189.
wherein the cannabinoids, together, are of greater than 1% wt-30% wt., and
wherein the composition comprises at two or more terpenes/two or more flavonoids selected from the group consisting of: sabinene hydrate, (−)-isopulegol fenchol, isoborneol, nerol, r-(+)-pulegone, geranyl acetate, and cedrol, are less than 0% wt.-30% wt. and
wherein the composition is an edible.

192. The composition according to claim 189.
wherein the combined cannabinoids, together, are more than 0% wt.-30% wt., and or
wherein the composition of two or more terpenes/two or more flavonoids selected from the group consisting of: sabinene hydrate, (−)-isopulegol, glycitein, malvidin, naringenin, fenchol, frenchone, malvidin, baicalein, isoborneol, nerol, r-(+)-pulegone, geranyl acetate, and cedrol, wherein the terpenes are greater 0% wt.-30% wt., and
wherein the composition is a Topical, Shampoos, cough drops, or pills.

193. The composition according to claim 189, further comprising: selected from the group consisting of: wherein a combination of terpenes and flavonoids are greater than 0% wt.-30% wt., and wherein the composition comprises of two or more terpenes/two or more Flavonoids selected from the group consisting of: sabinene hydrate; (−)-isospulegol, galangin, epicatechin, naringenin, chrysin, fenchol, isoborneol, nerol, r-(+)-pulegone, menthol, borneol, terpineol, fisetin, malvidin, glycitein, acetate, and cedrol, wherein the componds are greater than 0% wt. 30%. wt. and
0%-30% wt. alcohol
0% wt.-35 % wt. Hops

194. The composition according to claim 89, wherein the composition comprises:
0% cannabinoids, and
wherein composition comprises of two or more
terpenes/two or more Flavonoids and one or more Flavonols, one or more Flavones, are greater than 0% wt.-30% wt. selected from the group consisting of; sabinene hydrate, (−)-isopulegol fenchol, menthol, frenchone, Apigenin, Chysin, limonene, linalool, borneol, glycitein, malvidin, baicaiein, myrcene, Quercetin, Fisetin, camphene, nerol, sabinene, a pinene isoborneol, myrcene, terpineol nerol, r-(+)-pulegone, geranyl, borneol, eucalyptol, and sabinene wherein the composition is a Carbonated liquid and 0% wt.-30% wt. alcohol 0% wt.-35 % wt. Hops.
